(19) United States
(12) Patent Application Publication
(10) Pub. No.: US 2018/0311241 A1
(43) Pub. Date: Nov. 1, 2018

(54) PHARMACEUTICAL SYNERGISTIC COMBINATION

(71) Applicant: CADILA HEALTHCARE LIMITED, Ahmedabad, Gujarat (IN)

(72) Inventors: Mukul JAIN, Ahmedabad (IN); Kaushik BANERJEE, Ahmedabad (IN); Amit JOHARAPURKAR, Ahmedabad (IN); Arti PANDYA, Ahmedabad (IN); Vishal PATEL, Ahmedabad (IN)

(21) Appl. No.: 15/771,334

(22) PCT Filed: Oct. 20, 2016
(86) PCT No.: PCT/IB2016/056311

§ 371 Date: Apr. 26, 2018

(30) Foreign Application Priority Data
Oct. 29, 2015 (IN) 4103/MUM/2015

(51) Int. Cl. A61K 31/513

(52) U.S. Cl. CPC A61K 31/513; A61K 31/216; A61K 2300/00

(57) ABSTRACT

The present invention describes a synergistic composition comprising of Fimasartan or pharmaceutically effective salts thereof and a NEP inhibitor or pharmaceutically effective salts thereof for the treatment of cardiovascular and related disease. The present invention is directed to a synergistic combination comprising combinations of Fimasartan or suitable pharmaceutically effective salts thereof and a NEP inhibitor or pharmaceutically effective salts thereof in a single unit pharmaceutical composition. The present invention is further directed to combinations of Fimasartan or a pharmaceutically effective salt thereof and a NEP inhibitor or a pharmaceutically effective salt thereof. The invention also relates to combining separate pharmaceutical compositions of Fimasartan and a NEP inhibitor in kit form. The invention is further related to methods of preparing such pharmaceutical composition in separate in either a kit form containing both the active agents and methods of treating a subject with the same.

### FIELD OF THE INVENTION

The present invention describes a synergistic composition comprising of Fimasartan or pharmaceutically effective salts thereof and a NEP inhibitor or pharmaceutically effective salts thereof for the treatment of cardiovascular and related disease. The present invention is directed to a synergistic combination comprising combinations of Fimasartan or suitable pharmaceutically effective salts thereof and a NEP inhibitor or pharmaceutically effective salts thereof in a single unit pharmaceutical composition. The present invention is further directed to combinations of Fimasartan or a pharmaceutically effective salt thereof and a NEP inhibitor or a pharmaceutically effective salt thereof. The invention also relates to combining separate pharmaceutical compositions of Fimasartan and a NEP inhibitor in kit form. The invention is further related to methods of preparing such pharmaceutical composition in separate in either a kit form containing both the active agents and methods of treating a subject with the same.

### BACKGROUND OF THE INVENTION

Hypertension is one of the most common cardiovascular diseases which can lead to occurrence of acute and chronic heart disease, myocardial infarction, stroke, and heart failure. Hence there is a continued need for a therapeutic approach that would be effective antihypertensive agents and arrest the complications of hypertension like heart failure. Although a variety of drugs have been used for the purpose of treating conditions arising out of hypertension, they are not always successful (Waber B, American Journal of Hypertension 1997, 10 (7.2):131S-137S).

Renin-angiotensin aldosterone system regulates blood pressure by its action on cardiovascular and renal system by involvement of angiotensin 1 and 2 receptor (AT-1 and AT-2) (J Hum Hypertens. 1995 November; 9 Suppl 5:S19-24). Angiotensin II acts on angiotensin receptors of which AT-1 is of particular importance. Angiotensin II increases blood pressure by stimulating cardiac smooth muscle and stimulates sodium reabsorption in kidneys which leads to hypertension. Since, AT-1 receptor activation by angiotensin II drives hypertensive condition, blockade of AT-1 receptor can prevent and treat hypertension. Several such AT-1 receptor antagonists are used in clinical practice which include telmisartan, valsartan, losartan, irbesartan, azilsartan, olmesartan, saprisartan, tasosartan, or in each case a pharmaceutically acceptable salt thereof. Angiotensin I is converted to biologically active form angiotensin II by angiotensin converting enzyme (ACE). Many agents that inhibit ACE are also used as antihypertensive agents which include captopril, enalapril, lisinopril, benazepril and spirapril (Drugs. 1990; 39 Suppl 2:11-6). Apart from conversion of angiotensin I to angiotensin II, ACE involved in degradation of bradykinin, hence ACE inhibitor also show hypotensive effect by increasing bradykinin levels. Hence, it has been presumed that ACE inhibitor act not only through renin angiotensin aldosterone system but also by inhibiting degradation of bradykinin.

Neprilysin or NEP or Neural endoeptidase (also known as CD10, MME, CALLA, EC 3.4.24.11, enkaphalinase, atriopeptidase) is a most important membrane bound endopeptidase involved in the metabolism of various peptides and belong to the gluzincin family of metaloprotease having a Zn²⁺ ion as cofactor (Corvol and TA, 1998 and Corvol and Williams, 1998). NEP is present in various tissues like vascular endothelium, smooth muscle cells, brain, spinal cord, and peripheral nervous system. NEP is a 90-100 kDA cell surface peptidase that inactivates a variety of vasoactive peptides which include ANP (atrial natriuretic peptide or factor, also known as ANF), BNP (brain natriuretic peptide), glucagon, enkephalins, substance P, neurotensin, oxytocin, and bradykinin (J Surg Res 2005, 128(1):21-27, J Cardiovasc Pharmacol 2005, 46(3):390-397, Circulation 2002, 106(8):920-926). ANP is largely synthesized in the atria and BNP in the ventricles. (Journal of Cardiology (2011) 57, 131-140). ANP binds to NPR-A receptor and produces its biological actions via a cGMP-dependent pathway. ANP is a vasodilator, diuretic and natriuretic agents, released by heart in response to high blood volume. Its main function is to lower blood pressure and to control electrolyte homeostasis. Kidney and CVS is the main target organs of ANP but ANP also interacts with many other hormones in order to regulate their secretion.

Three mechanisms are mainly involved in the inactivation or degradation of ANP which include: 1) receptor-mediated degradation, 2) degradation by extracellular proteases (NEP), and 3) secretion of the peptides into body fluids such as urine or bile (FEBS Journal 278 (2011) 1808-1817). Several reports indicated that NEP inhibitor elevates natriuretic peptide concentrations in humans and animals, and increased sodium excretion during heart failure (Br J Pharmacol 113, 204-208., Hypertension 30, 184-190, Physiol 271, R373-R380, Circulation 91, 2036-2042., Clin Exp Hypertens 17, 861-876.). It has been reported that NEP inhibitor, candoxatrilat potentiates natriuretic and cyclic GMP responses to a low-dose ANP infusion. (Br J Pharmacol. 1992 September; 107(1):50-7.)

To achieve a successful therapeutic goal, in the prior art, arbitrary selection of various classes of antihypertensive agents is used which does not always provide the desired therapeutic goal. Hence, a new therapeutic approach with enhanced efficacy, lesser side effects, and better prevention and treatment of heart failure is needed. The hypertensive condition can damage blood vessels which lead to end organs damage. The higher the blood pressure and the longer it goes uncontrolled, the greater the damage to the organs. Uncontrolled high blood pressure for longer duration can lead to heart attack or stroke, weakened and narrowed blood vessels in the kidneys, thickened, narrowed or torn blood vessels in the eyes and metabolic syndrome. Hence it is important to achieve additional cardiovascular protection than mere reduction in blood pressure during treatment of hypertension (Am J Cardiovasc Dis 2012; 2(3):160-170).

Numerous factors are associated with development of hypertension, which includes intrauterine malnutrition, family history of hypertension, obesity, particularly excess abdominal fat, insulin resistance, high dietary sodium intakes, low dietary intakes of calcium, potassium and magnesium, physical inactivity, high alcohol intakes, tobacco use, drug use (e.g., cocaine, ecstasy, anabolic steroids), emotional stress, diet pill use, oral contraceptives (Pediatr Clin North Am 1999; 46(2):235-252, Circulation 2000; 101(3):329-335, Environ Health Perspect 2000; 108 Suppl 3:545-553). Hence, it has been concluded that nature of hypertensive vascular disease is multifactorial. Hence it is very difficult for single drug treatment to maintain constant or near-constant BP in response to various stressors. BP is primarily determined by 3 major factors: renal sodium excretion and resultant plasma and total body volume, cardiac performance, and vascular tone. These factors control intravascular volume, cardiac output, and systemic vascular resistance, which are the immediate hemodynamic determinants of BP. Though, a specific cause for hypertension can be identified in some patients, hypertension is usually multifactorial, making it very difficult to normalize it by interfering with only a single presser mechanism. Furthermore, drug treatment directed at any one factor routinely evokes a compensatory (counter regulatory) response that finally reduces the magnitude of response, even if it is precisely directed at the predominant pathophysiologic mechanism involved in hypertension. As a consequence, limited blood pressure reduction is seen with majority of the currently available approaches. Hence combination approach is necessary for complete treatment of diseases arising out of hypertension. But combination approach is useful only when, the combination effect is additive, with deleterious side effects, and has additional benefit on additional cardiovascular end points (J Clin Hypertens (Greenwich). 2011; 13:146-154, Curr Opin Nephrol Hypertens. 2012 September; 21(5):486-91).

Hence, there is a continuous need for further development of therapeutic methods and combinations for the treatment of hypertension and diseases arising out of hypertension.

Fimasartan which is chemically defined as 2-n-butyl-5-dimethylaminothiocarbonyl-methyl-6-methyl-3-[[2′-(1H-tetrazol-5-yl)biphenyl-4-yl]methyl]pyrimidin-4(3H)-one, has the following structural formula:

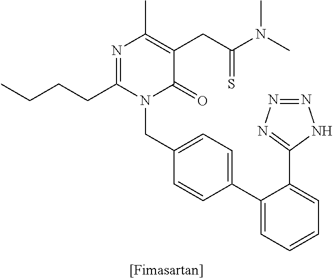 [SMILES: CCCCc1nc(C)c(CC(=S)N(C)C)c(=O)n1Cc1ccc(-c2ccccc2-c2nnn[nH]2)cc1]

It is an antihypertensive agent of the AT-1 receptor antagonist or blocker class and has currently been approved as a pharmaceutical product under brand name of KANARB®, in patients with mild to moderate essential hypertension, Fimasartan, at a dose of 60 mg to 120 mg, demonstrated antihypertensive effects in terms of changes in diastolic blood pressure (DBP) in the sitting position at Week 12 of drug administration (Lee S E, Clin Ther. 2012; 34(3):552-68-68 e1-9).

Neutral endopeptidase (NEP) inhibitors are disclosed in U.S. Pat. No. 4,722,810, U.S. Pat. No. 5,223,516, U.S. Pat. No. 4,610,816, U.S. Pat. No. 4,929,641, South African Patent Application 84/0670, UK 69578, U.S. Pat. No. 5,217,996, EP 00342850, GB 02218983, WO 92/14706, EP 00343911, JP 06234754, EP 00361365, WO 90/09374, JP 07157459, WO 94/15908, U.S. Pat. No. 5,273,990, U.S. Pat. No. 5,294,632, U.S. Pat. No. 5,250,522, EP 00636621, WO 93/09101 EP 00590442, WO 93/10773, U.S. Pat. No. 5,217,996, the disclosure of each of which is incorporated by reference. Neutral endopeptidase inhibitors may be used for purposes of this invention either in their free form, as well as in any suitable salt form. Reference to neutral endopeptidase inhibitors includes reference to their pharmaceutically acceptable salts also.

Sacubitril, also known as, AHU-377, (4-[[(2S,4R)-5-ethoxy-4-methyl-5-oxo-1-(4-phenylphenyl)pentan-2-yl]amino]-4-oxobutanoic acid, C24H29NO5, MW 411.49) is an inhibitor of neprilysin, having the following structure:

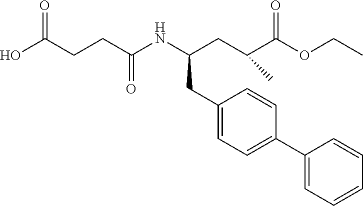 [SMILES: CCOC(=O)[C@H](C)C[C@@H](Cc1ccc(-c2ccccc2)cc1)NC(=O)CCC(=O)O]

The combination drug, Valsartan/Sacubitril is known as LCZ696 and marketed under the brand name, Entresto for treatment of heart failure.

The present invention is intended to provide an antihypertensive pharmaceutical composition which is effective not only for the prevention, alleviation and treatment of hypertension, but also for the prevention, alleviation or treatment of complications of hypertension and/or diabetes and hyperlipidemia. Sacubitril or its pharmaceutically acceptable salt or a solvate thereof or a hydrate thereof used in the combination may be either in crystalline or amorphous form and the Fimasartan or its pharmaceutically acceptable salt thereof or a solvate thereof or a hydrate thereof used herein may also be in crystalline or amorphous form. Such a synergistic composition may be more effective as an antihypertensive therapy (whether for malignant, essential, reno-vascular, diabetic, isolated systolic, or other secondary type of hypertension) through improved efficacy as well as a greater responder rate than when used either alone or in combination with existing therapy. The combinations of Sacubitril or its pharmaceutically acceptable salt thereof or a solvate thereof or a hydrate thereof and Fimasartan or its pharmaceutically acceptable salt thereof or a solvate thereof or a hydrate thereof may be in crystalline or amorphous and can also be useful in the treatment or prevention of heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter or detrimental vascular remodeling. The combination is also useful in treating atherosclerosis, angina (whether stable or unstable), and renal insufficiency (diabetic and non-diabetic). Combination of Sacubitril and Fimasartan as disclosed herein, may also have potential to improve endothelial dysfunction, thereby providing benefit in diseases in which normal endothelial function is disrupted such as heart failure, angina pectoris and diabetes. Furthermore, the combination of the present invention can be useful for the treatment or prevention of secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke. prevention of, delay the onset of and/or treatment of hypertension (whether for malignant, essential, reno-vascular, diabetic, isolated systolic, or other secondary type of hypertension), heart failure such as diastolic and congestive heart failure (acute and chronic), left ventricular dysfunction, sinus node dysfunction, Arrhythmia (bradyarrthmia or tacharrhythmia), hypertensive carotid sinus, bifascicular and trifascicular block, mitral valve prolapse, Aortic regurgitation, cardiac tumor, aortic occlusion, atheroembolism, thrombosis, endothelial dysfunction, diastolic dysfunction, hypertrophic cardiomyopathy, diabetic cardiac myopathy, supraventricular and ventricular arrhythmias, atrial fibrillation (AF), cardiac fibrosis, atrial flutter, detrimental vascular remodeling, plaque stabilization, myocardial infarction (Ml) and its sequelae, atherosclerosis including coronary arterial disease (CAD), Dextrocardia, Rhabdomyoma, Cardiomyopathy, peripheral pulmonic stenosis, Aortic incompetence, Arterial dialation and rupture, mitral regurgitation, sinus node dysfunction, Bradycardia or tachycardia, hypertensive carotid sinus and neurovascular syndrome, aortic regurgitation, pericarditis, cardiac tumor, aortic occlusion, Asymmetric septal hypertrophy, dyspnea, Cardiac edema, angina pectoris (whether unstable or stable), renal insufficiency (diabetic and non-diabetic), renal fibrosis, polycystic kidney disease (PKD), type 2 diabetes, metabolic syndrome, secondary aldosteronism, primary and secondary pulmonary hypertension, renal failure conditions such as nephrotic syndrome, diabetic nephropathy, glomerulonephritis, scleroderma, glomerular sclerosis, proteinuria of primary renal disease, renal vascular hypertension, diabetic retinopathy and end-stage renal disease (ESRD), the management of other vascular disorders such as migraine, peripheral vascular disease (PVD), Raynaud's disease, luminal hyperplasia, cognitive dysfunction (such as Alzheimer's), glaucoma and cerebrovascular disease such as embolic or thrombotic stroke.

In the present invention, the Fimasartan used may be used as the Fimasartan potassium salt, more preferably as Fimasartan potassium trihydrate. This compound is commercially available or otherwise may be prepared by using a known method. In another embodiment, other pharmaceutically acceptable salts of Fimasartan may be used, some of which may be novel. The present invention therefore also discloses certain novel salts of Fimasartan. In the present invention, the neprilysin inhibitor is preferably Sacubitril and its pharmaceutically acceptable salt. This materials are commercially available or otherwise may be prepared by using a known method. The composition of the present invention may contain an AT-1 receptor blocker Fimasartan in amount of 0.5 to 240 mg. Further, the composition of the present invention may contain a neprilysin inhibitor Sacubitril in an amount of 0.1 to 400 mg.

The composition of the present invention exhibits an enhanced antihypertensive effect greater than the simple sum of antihypertensive values of the same doses of individual active ingredients. Accordingly, the composition of the present invention enables the use of individual active ingredients at a content or dose lower than that upon separate use thereof, and thus can provide more effective treatment or prevention of hypertension or the like while reducing adverse side effects due to an overdose of individual active ingredients.

### EMBODIMENTS OF THE PRESENT INVENTION

In a first aspect, the present invention relates to pharmaceutical combinations comprising Fimasartan or a pharmaceutically effective salt thereof and one or more NEP inhibitors or a pharmaceutically effective salt thereof, optionally in the presence of suitable pharmaceutically acceptable carriers and pharmaceutical compositions comprising them.

The invention also relates to combining separate pharmaceutical compositions of Fimasartan and a NEP inhibitor in a kit form.

In another aspect, the present invention provides a synergistic composition comprising a low dose of Fimasartan or a pharmaceutically effective salt thereof and NEP inhibitor or a pharmaceutically effective salt thereof as described herein for the treatment of hypertension and related diseases.

In a further aspect, the present invention provides a synergistic composition comprising a low dose of Fimasartan or pharmaceutically effective salts thereof with a NEP inhibitor or a pharmaceutically effective salt thereof as described herein for the treatment of humans. In an embodiment, the preferred NEP inhibitor may be selected from Sacubitril.

In another embodiment is provided a pharmaceutical composition containing effective amount of synergistic composition suitable for treatment of hypertension and related diseases.

In yet another embodiment is provided a process for the preparation such pharmaceutical compositions of the synergistic composition or compositions in a separate kit form and methods of treating a subject with the same:

### DETAILED DESCRIPTION OF THE INVENTION

In a first aspect, the present invention relates to pharmaceutical combinations comprising Fimasartan or pharmaceutically effective salts thereof with a NEP inhibitor or a pharmaceutically effective salt thereof, optionally in the presence of atleast a pharmaceutically acceptable carrier and pharmaceutical compositions comprising them.

In a preferred embodiment, the NEP inhibitor may be selected from Sacubitril (4-[[(2S,4R)-5-ethoxy-4-methyl-5-oxo-1-(4-phenylphenyl)pentan-2-yl]amino]-4-oxobutanoic acid

Accordingly, in one aspect the present invention also provides a pharmaceutical composition comprising Fimasartan or a pharmaceutically effective salt thereof and a NEP inhibitor or a pharmaceutically effective salt thereof and one or more suitable pharmaceutically acceptable carriers.

Thus, in a further aspect, the invention also provides a process for preparing a pharmaceutical composition comprising Fimasartan or pharmaceutically effective salt thereof and a NEP inhibitor or a pharmaceutically effective salt thereof and a pharmaceutically acceptable carrier thereof, which process comprises admixing the Fimasartan or a pharmaceutically effective salt thereof and NEP inhibitor or a pharmaceutically effective salt thereof and atleast one pharmaceutically acceptable carrier.

The pharmaceutically acceptable carrier may be selected from sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrates, buffers, preservatives, antioxidants, lubricants, flavoring agents, and the like commonly used in pharmaceutical formulations.

The compositions are preferably in a unit dosage form in an amount appropriate for the relevant daily dosage.

As used herein the term ‘pharmaceutically acceptable’ embraces both human and veterinary use: for example the term ‘pharmaceutically acceptable’ embraces a compound useful as a veterinary drug.

In the treatment the medicaments may be administered from 1 to 6 times a day, but most preferably 1 or 2 times per day.

Also, the dosages of each particular active agent in any given composition can, as required, vary within a range of doses known to be required in respect of accepted dosage regimens for that compound. Dosages of each active agent can also be adapted as required to take into account advantageous effects of combining the agents as mentioned herein.

In particular, the present invention provides a pharmaceutical composition comprising Fimasartan or a pharmaceutically effective salt thereof and a NEP inhibitor or a pharmaceutically effective salt thereof and suitable pharmaceutically acceptable carrier thereof, for use in the treatment of hypertension conditions associated with them.

The dosage of the active compound can depend on a variety of factors, such as mode of administration, homeothermic species, age and/or individual condition.

The combination of NEP inhibitor or a pharmaceutically acceptable salt thereof and fimasartan or a pharmaceutically acceptable salt thereof is used at a dose of about 1 mg to about 1000 mg.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e. g. for a patient of approximately 75 kg in weight. Fimasartan is supplied in the form of suitable dosage unit form, for example, a capsule or tablet, and comprising a therapeutically effective amount, e. g. from about 20 to about 320 mg, of Fimasartan which may be applied to patients. The application of the active ingredient may occur up to three times a day, starting e. g. with a daily dose of 2.0 mg or 40 mg of Fimasartan, increasing via 80 mg daily and further to 160 mg daily up to 320 mg daily. Preferably, Fimasartan is applied once a day or twice a day in heart failure patients with a dose of 80 mg or 160 mg, respectively.

In case of NEP inhibitors, preferred for Sacubitril, preferred dosage unit forms are, for example, tablets or capsules comprising e. g. from about 20 mg to about 800 mg, preferably from about 50 mg to about 700 mg, even more preferably from about 100 ma to about 600 mg and even more preferably from about 100 mg to about 300 mg, administered once a day.

Usually the compositions are adapted for oral administration. However, they may be adapted for other modes of administration, for example parenteral administration, sublingual or transdermal administration.

The compositions may be in the form of tablets, capsules, powders, granules, lozenges, suppositories, reconstitutable powders, or liquid preparations, such as oral or sterile parenteral solutions or suspensions.

In order to obtain consistency of administration it is preferred that a composition of the invention is in the form of a unit dose.

Unit dosage presentation forms for oral administration may be in tablet or capsule form and may as necessary contain conventional excipients such as binding agents, fillers, lubricants, glidants, disintegrates and wetting agents.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, liquid dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anesthetic, a preservative and buffering agent can be dissolved in the vehicle. To enhance the stability, the composition can be frozen after filling into the vial and the water removed under vacuum. Parenteral suspensions are prepared in substantially the same manner, except that the active compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Examples of binding agents include acacia, alginic acid, carboxymethylcellulose calcium, carboxymethylcellulose sodium, dextrates, dextrin, dextrose, ethylcellulose, gelatin, liquid glucose, guar gum, hydroxyethyl cellulose, hydroxypropyl cellulose, hydroxypropyl methylcellulose, magnesium aluminium silicate, maltodextrin, methyl cellulose, polymethacrylates, polyvinylpyrrolidone, pregelatinised starch, sodium alginate, sorbitol, starch, syrup, tragacanth.

Examples of fillers include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol.

Examples of lubricants include calcium stearate, glyceryl monostearate, glyceryl palmitostearate, magnesium stearate, microcrystalline cellulose, sodium benzoate, sodium chloride, sodium lauryl sulphate, stearic acid, sodium stearyl fumarate, talc, zinc stearate.

Examples of glidants include colloidal silicon dioxide, powdered cellulose, magnesium trisilicate, silicon dioxide, talc.

Examples of disintegrants include alginic acid, carboxymethylcellulose calcium, carboxymethylcellulose sodium, colloidal silicon dioxide, croscarmellose sodium, crospovidone, guar gum, magnesium aluminium silicate, microcrystalline cellulose, methyl cellulose, polyvinylpyrrolidone, polacrilin potassium, pregelatinised starch, sodium alginate, sodium lauryl sulphate, sodium starch glycollate.

An example of a pharmaceutically acceptable wetting agent is sodium lauryl sulphate.

The compositions are prepared and formulated according to conventional methods, such as those disclosed in standard reference texts and are well within the scope of a skilled person. For example, the solid oral compositions may be prepared by conventional methods of blending, filling or tableting. Repeated blending operations may be used to distribute the active agent throughout those compositions employing large quantities of fillers. Such operations are of course conventional in the art. The tablets may be coated according to methods well known in normal pharmaceutical practice.

Compositions may, if desired, be in the form of a pack accompanied by written or printed instructions for use.

No adverse toxicological effects were seen for the compositions or methods of the invention in the above mentioned dosage ranges. Further the composition of the present invention was found suitable for the treatment of hypertension and its associated disorders.

The invention also relates to combining separate pharmaceutical compositions in kit form. That is a kit combining two separate units: Fimasartan or pharmaceutically effective salts thereof along with a NEP inhibitor or a pharmaceutically effective salt thereof. The kit form is particularly advantageous when the separate components must be administered in different dosage forms or are administered at different dosage intervals.

These pharmaceutical preparations are for enteral, such as oral, and also rectal or parenteral, administration to homeotherms with the preparations comprising the pharmacological active compound either alone or together with customary pharmaceutical auxiliary substances. For example, the pharmaceutical preparations consist of from about 0.1% to 90%, preferably of from about 1% to about 80%, of the active compounds.

Pharmaceutical preparations for enteral or parenteral administration are, for example, in unit dose forms, such as coated tablets, tablets, capsules or suppositories and also ampoules.

These are prepared in a manner which is known per se, for example using conventional mixing, granulation, coating, solubilizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homoeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e. g. for a patient of approximately 75 kg in weight.

In a further aspect, the present invention is provided a synergistic composition comprising a low dose of compound of Fimasartan or pharmaceutically effective salts thereof with a NEP inhibitor or a pharmaceutically effective salt thereof for the treatment of hypertension and related diseases

### Biological Studies:

### Experimental Investigation for Antihypertensive Effects

This study is intended to examine antihypertensive action of individual drugs. For this purpose, test drugs, i.e., Fimasartan or its pharmaceutically acceptable salt, and Sacubitril or its pharmaceutically acceptable salt, or a combination of Fimasartan or its pharmaceutically acceptable salt, and Sacubitril or its pharmaceutically acceptable salt, with or without a pharmaceutical career will be orally administered for 4 weeks to rats with hypertension. Changes in blood pressure, heart rate, body weight, and metabolic profile of animals were measured to investigate the antihypertensive and metabolic action by long-term administration of individual drugs, confirm the maintenance degree of effective and stable blood pressure, and examine the difference between single administration and combined administration of drugs. In this manner, the antihypertensive action will be compared and evaluated between the individual drugs.

### Experimental Animals Models

### Aortocaval Fistula-Induced Congestive Heart Failure and Cardiac Hypertrophy

Sprague Dawley rats will be used in the study at the age to 6-8 weeks. A midline abdominal incision will be made under anesthesia to expose the vena cava and abdominal aorta distal to the origin of the renal arteries. A longitudinal incision will be performed in the outer wall of the vena cava. The common wall between the aorta and vena cava will be grabbed through the incision and a fistula (1.0-1.2 mm outer diameter) will be created between the vena cava and aorta. The opening of the outer wall of the vena cava will be closed with a continuous suture. After the surgical procedure, the animals will be allowed to recover with daily monitoring of urine output and sodium excretion in metabolic cages. A matched group of sham-operated rats will serve as controls. After a week of surgical operation, rats will be divided into subgroups according to their daily absolute rate of sodium excretion. Fimasartan or its pharmaceutically acceptable salt, or Sacubitril or its pharmaceutically acceptable salt or their combination will be taken as a low dose, middle dose, and high dose group. Animals will be treated daily for four weeks with weekly measurement of blood pressure. Additionally, sodium excretion and creatinine clearance will be measured twice weekly.

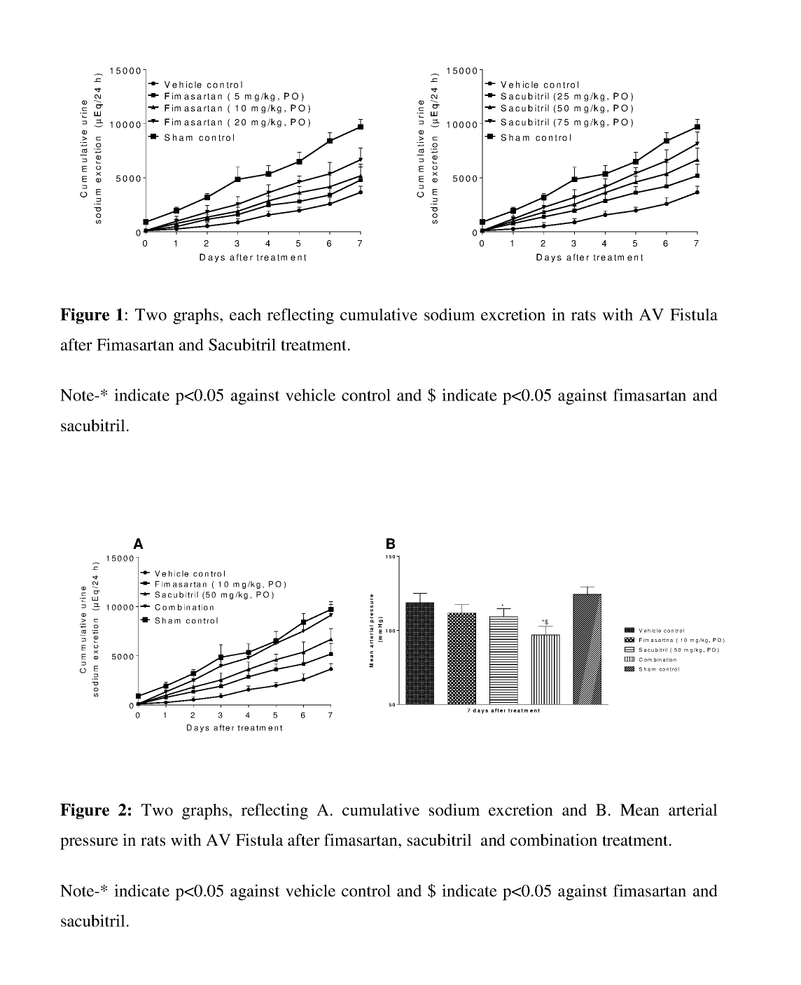

In the rat model of infrarenal abdominal aorta-vena cava fistula (AV fistula), workload on heart increases by sustained volume overload, this reduces sodium excretion and leads to cardiac hypertrophy. Rats receiving fimasartan (5, 10 and 30 mg/kg/day), sacubitril (25, 50 and 75 mg/kg/day) and combination (fimasartan 10 mg/kg and sacubitril 50 mg/kg) were evaluated in AV fistula model. Sprague Dawley rats in these experimental groups were treated daily by gavage for seven days and urine volume and sodium excretion were daily measured. Rats treated with vehicle served as controls. Rats with sham operation or animals with arterio-venous fistula treated with vehicle served as controls. Animals were randomized based on 24 h urinary sodium excretion. Animals with <100 μEq/24 h urinary sodium excretion were selected in study except sham control animals. Treatment was initiated for 7 days with daily measurement of sodium excretion and results were expressed as cumulative urinary sodium excretion. Vehicle control showed reduced cumulative urinary sodium excretion than sham control (166.0±12.5% against vehicle control) after seven days of AV Fistula. As shown in FIG. 1, chronic administration of fimasartan and sacubitril showed dose related increase in cumulative urinary sodium excretion rate (31.1±13.6, 41.9±12.2 and 82.6±13.1% at 5, 10 and 20 mg/kg of fimasartan and 41.9±12.1, 82.6±13.5 and 150.1±10.4% in 25, 50 and 75 mg/kg of sacubitril against vehicle control). Combination of fimasartan and sacubitril, showed significantly synergistic effect on cumulative urinary sodium excretion (150.1±10.4% against vehicle control) than alone fimasartan and sacubitril (FIG. 2). These observations indicate that combination of fimasartan and sacubitril reduce volume overload associated with AV Fistula. After completion of the treatment period, mean arterial blood pressure was evaluated from animals from different experimental groups. Further, vehicle control showed slightly reduced mean arterial blood pressure than sham control (4.8±1.7% rise in comparison to vehicle control). Fimasartan (5.9±1.9%) and sacubitril (8.0±1.9%) further reduced mean arterial blood pressure, while combination showed synergistic reduction (18.4±2.1%) than fimasartan or sacubitril alone (FIG. 2).

TABLE 1
Effect of fimasartan on cumulative sodium excretion
in rats after 7 days of treatment.

| | Urinary | % change in | Statistical |
|---|---|---|---|
| Treatment Group | sodium(μeq/24 h) | urinary sodium | significance |
| Vehicle control | 3649.8 ± 229.6 | | |
| Fimasartan | 4784.8 ± 497.5* | 31.1 ± 13.6* | P < 0.05 |
| (5 mg/kg, PO) | | | |
| Fimasartan | 5179.7 ± 451.2 | 41.9 ± 12.2* | P < 0.05 |
| (10 mg/kg, PO) | | | |
| Fimasartan | 6664.7 ± 448.2 | 82.6 ± 13.1* | P < 0.05 |
| (20 mg/kg, PO) | | | |
| Sham control | 9709.5 ± 468.2 | 166 ± 12.5* | P < 0.05 |

Note-
*indicate p < 0.05 against vehicle control.

TABLE 2
Effect of Sacubitril on cumulative sodium excretion
in rats after 7 days of treatment.

| | Urinary | % change om | Statistical |
|---|---|---|---|
| Treatment Group | sodium(μeq/24 h) | urinary sodium | significance |
| Vehicle control | 3649.8 ± 229.6 | | |
| Sacubitril | 5185.7 ± 443.7 | 41.9 ± 12.2* | P < 0.05 |
| (25 mg/kg, PO) | | | |
| Sacubitril | 6678.7 ± 451.7 | 82.6 ± 13.5* | P < 0.05 |
| (50 mg/kg, PO) | | | |
| Sacubitril | 9129.7 ± 449.7 | 150.1 ± 10.4* | P < 0.05 |
| (75 mg/kg, PO) | | | |
| Sham control | 9709.5 ± 457.1 | 166 ± 12.5* | P < 0.05 |

Note-
*indicate p < 0.05 against vehicle.

TABLE 3
Effect of fimasartan, sacubitril and combination on cumulative
sodium excretion in rats after 7 days of treatment.

| | Urinary | % change om | Statistical |
|---|---|---|---|
| Treatment Group | sodium(μeq/24 h) | urinary sodium | significance |
| Vehicle control | 3649.8 ± 229.6 | | |
| Fimasartan | 5179.7 ± 443.7 | 51.2 ± 11.9* | P < 0.05 |
| (10 mg/kg, PO) | | | |
| Sacubitril | 6678.7 ± 451.7 | 82.6 ± 13.5* | P < 0.05 |
| (50 mg/kg, PO) | | | |
| Combination | 9129.7 ± 452.2 | 150.1 ± 14.2*$ | P < 0.05 |
| Sham control | 9709.5 ± 468.2 | 166 ± 12.5* | P < 0.05 |

Note-
*indicate p < 0.05 against vehicle control and
$ indicate p < 0.05 against fimasartan and sacubitril

TABLE 3
Effect of fimasartan, sacubitril and combination on mean
arterial pressure in rats after 7 days of treatment.

| | | % change | Statis- |
|---|---|---|---|
| | Mean arterial | in meal | tical |
| | pressure | arterial | signifi- |
| Treatment Group | (mmHg) | pressure | cance |
| Vehicle control | 118.7 ± 2.6 | | |
| Fimasartan (10 mg/kg, PO) | 111.7 ± 2.3 | −5.9 ± 1.9* | P < 0.05 |
| Sacubitril (50 mg/kg, PO) | 109.2 ± 2.2 | −8 ± 1.9* | P < 0.05 |
| Combination | 96.8 ± 2.5 | −18.4 ± 2.1*$ | P < 0.05 |
| Sham control | 124.3 ± 2.1 | 4.8 ± 1.7* | P < 0.05 |

Note-
*indicate p < 0.05 against vehicle control and
$ indicate p < 0.05 against fimasartan and sacubitril

As shown in FIG. 1, chronic administration of fimasartan and sacubitril showed dose related increase in cumulative urinary sodium excretion rate (31.1±13.6, 41.9±12.2 and 82.6±13.1% at 5, 10 and 20 mg/kg of fimasartan and 41.9±12.1, 82.6±13.5 and 150.1±10.4% in 25, 50 and 75 mg/kg of sacubitril against vehicle control). Combination of fimasartan and sacubitril, showed significantly synergistic effect on cumulative urinary sodium excretion (150.1±10.4% against vehicle control). Fimasartan (5.9±1.9%) and sacubitril (8.0±1.9%) further reduced mean arterial blood pressure, while combination showed synergistic reduction (18.4±2.1%) than fimasartan or sacubitril alone (FIG. 2). So, from the result it shows combination of fimasartan and sacubitril, showed significantly synergistic effect as compared to alone fimasartan and sacubitril.

### Subtotal Nephrectomy in Rats

### Subtotal Nephrectomy Induced Hypertension

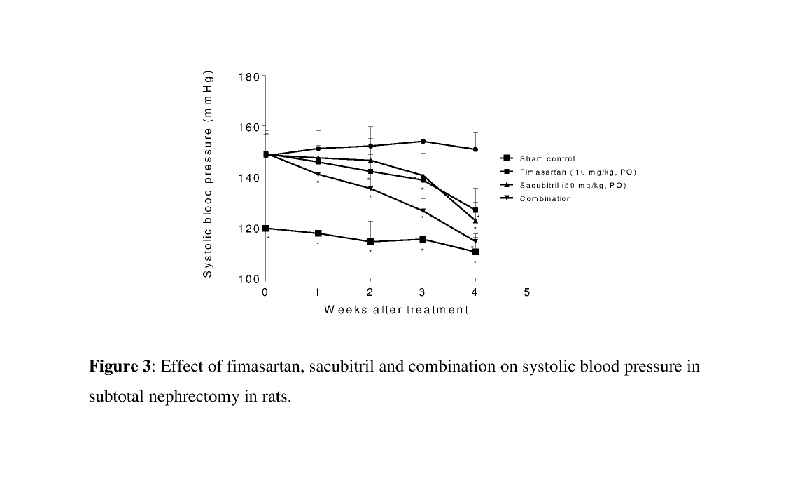

Fimasartan 10 mg/kg and Sacubitril 50 mg/kg and combination were administered for 4 weeks. Prior to initiation, Sprague Dawley rats were randomized based on their systolic blood pressure after one week of recovery. These experimental groups were treated daily by gavage for four weeks and their weekly blood pressure was measured. Rats treated with vehicle served as controls and sham operated animals treated with vehicle served as sham controls. Vehicle control showed rise in blood pressure (26.8%) in comparison to sham control. Fimasartan and sacubitril showed significant reduction (15.9 and 18.7% reductions against vehicle control), while combination normalize (24.0% against vehicle control) rise in blood pressure four weeks of treatment (FIG. 3).

Experimental model of acute cardiac failure is characterized by several features that closely mimic the pathophysiological consequences of CHF in patients, includes the characteristic neurohumoral activation, decrease in renal perfusion, sodium retention and cardiac hypertrophy (J Biomed Biotechnol. 2011; 2011: 729497). Present study demonstrates that acute administration of combination of sacubitril and fimasartan produced significant diuretic and natriuretic responses along with a remarkable decline in blood pressure. Five sixth nephrectomy represent a model of hypertension in rats and in clinic it has been observed that loss of renal mass is associated with increased hypertension (APMIS. 1992 December; 100(12):1097-105; International Journal of Urology (2015) 22, 797-804). Current results indicate that combination of sacubitril and fituasartan can suppresses rise in blood pressure in five sixth nephrectomized rats.

1. A pharmaceutical composition comprising (i) the Fimasartan or a pharmaceutically effective salt thereof (ii) a NEP inhibitor or a pharmaceutical acceptable salt thereof and a pharmaceutically acceptable carrier.

2. The pharmaceutical composition as claimed in claim 1, wherein the NEP inhibitor is (4-[[2S,4R)-5-ethoxy-4-methyl-5-oxo-1-(4-phenylphenyl)pentan-2-yl]amino]-4-oxobutanoic acid or a pharmaceutically acceptable salt thereof.

3. The pharmaceutical composition of claim 1, wherein the fimasartan or the NEP inhibitor or pharmaceutically acceptable salt thereof is present at a dosage ranging from 1 mg to 1000 mg.

4. The pharmaceutical composition as claimed in claim 3, wherein the fimasartan is present at a dosage ranging from 40 mg to 320 mg and the NEP inhibitor or pharmaceutically acceptable salt thereof is present at a dosage ranging from 20 mg to 800 mg.

5. A kit comprising at least two separate containers in a single package pharmaceutical compositions comprising in one container a pharmaceutical composition comprising a NEP inhibitor and in a second container a pharmaceutical composition comprising fimasartan.

6. A solid oral dosage comprising moieties of fimasartan or a pharmaceutically acceptable salt thereof and 4-[[(2S,4R)-5-ethoxy-4-methyl-5-oxo-1-(4-phenylphenyl)pentan-2-yl]amino]-4-oxobutanoic acid or a pharmaceutically acceptable salt thereof in a concentration from about 4% to about 90% by weight of the composition; and at least one pharmaceutically acceptable excipient.
